(19) United States
(12) Patent Application Publication
(10) Pub. No.: US 2014/0093543 A1
(43) Pub. Date: Apr. 3, 2014

(54) METHOD AND VACUUM CONTAINER FOR PREPARING A BONE GRAFT

(71) Applicant: Vittorio M. Morreale, Rochester, MI (US)

(72) Inventor: Vittorio M. Morreale, Rochester, MI (US)

(21) Appl. No.: 13/633,177

(22) Filed: Oct. 2, 2012

(51) Int. Cl. A61K 9/00; A61K 35/12; B05D 5/00; A61K 35/32; A61P 19/08; F04B 49/00; A61K 38/18

(52) U.S. Cl. 424/400; 137/565.11; 424/93.7; 514/8.8; 424/549; 427/2.26

(57) ABSTRACT

A vacuum container supports a cancellous device fully immersed within a solution of a bone growth promoting substance in a sealed chamber. The solution may include one of a Demineralized Bone Matrix (DBM) solution, a morphegenic protein solution, or a stem cell solution. A vacuum creating device is in fluid communication with the sealed chamber, and is operable to remove air from within the sealed chamber to create a vacuum therein. Removing air from the sealed chamber also removes air from a plurality of voids in the cancellous device, which allows the solution to fill the voids and saturate the cancellous device in preparation for use as a bone graft.

### TECHNICAL FIELD

The invention generally relates to a method and vacuum container for preparing a cancellous device for use as a bone graft.

### BACKGROUND

Bone grafting is a surgical procedure that places new bone or a replacement material into spaces between or around broken bone (fractures) or in holes in bone (defects) to aid in healing. Bone grafting is used to repair bone fractures that are extremely complex, pose a significant risk to the patient, or fail to heal properly. Bone grafting is also used to help fusion between vertebrae, correct deformities, or provide structural support for fractures of the spine. In addition to fracture repair, bone grafting is used to repair defects in bone caused by congenital disorders, traumatic injury, or surgery for bone cancer. Bone grafts are also used for facial or cranial reconstruction.

The term “graft” commonly refers to an autograft or allograft. A graft made of bone from the patient's own body (e.g., hip bones or ribs) is an autograft. An allograft uses bone from a cadaver, which has been frozen and stored in a tissue bank. Synthetic bone material may also be used as a graft. To place a bone graft, a surgeon makes an incision in the skin over the bone defect, and shapes the bone graft or replacement material to fit into it. After the graft is placed into the defect, it is held in place with pins, plates, or screws.

In surgery of the spine, especially spinal fusion (also called arthrodesis), surgeons may decide to use bone grafts to assist in the healing and remodeling of the spine after surgery. Normally, small pieces of bone are placed into the space between the vertebrae to be fused, and sometimes larger solid pieces of bone provide immediate structural support. Spinal fusion involves the surgical treatment of abnormalities in the vertebrae, such as curvatures, scoliosis or kyphosis, or injuries (fractures). For example, cervical spinal fusion joins selected bones in the neck. Bone grafts may be used for many different procedures, including but not limited to spinal fusion surgery, orthopedics, podiatry, dentistry, etc.

Demineralized Bone Matrix (DBM) is allograft bone that has had the inorganic mineral removed, leaving behind the organic collagen matrix. Removal of the bone mineral exposes more biologically active bone morphogenetic proteins. These growth factors modulate the differentiation of progenitor cells into osteoprogenitor cells, which are responsible for bone and cartilage formation, thereby promoting bone growth. Bone tissue is a porous, matrix-like structure. The success of a bone graft is determined by its ability to recruit host cells to the site of the graft and modulate their conversion into bone forming cells.

### SUMMARY

A method of preparing a bone graft is provided. The method includes immersing a cancellous device defining a plurality of voids within a solution. Air from the plurality of voids in the cancellous device is then removed while the cancellous device is immersed in the solution. Removing the air from the voids of the cancellous device allows the solution to fill the voids and saturate the cancellous device.

A method of saturating a cancellous device with a solution of Demineralized Bone Matrix (DBM) for use as a bone graft is also provided. The method includes placing the cancellous device and the solution of DBM within a reservoir of a container, with the cancellous device fully immersed within and surrounded by the solution of DBM. The container is then sealed to define a sealed chamber, with the cancellous device and the solution of DBM disposed within the sealed chamber. Air from the sealed chamber is then removed to form a vacuum in the sealed chamber. Forming the vacuum in the sealed chamber removes air trapped within a plurality of voids of the cancellous device, so that the solution of DBM may fill the plurality of voids and saturate the cancellous device.

A vacuum container for saturating a cancellous device with a solution for use as a bone graft is also provided. The vacuum container includes a lower portion defining a reservoir. The reservoir is configured for containing the cancellous device fully immersed within the solution. An upper portion is removably attached to the lower portion, and disposed in sealing engagement with the lower portion to define a sealed chamber. The upper portion defines an opening into the sealed chamber. A valve is coupled to the upper portion. The valve is operable to open and close fluid communication through the opening. A vacuum creating device is in fluid communication with the valve, and is operable to remove air from within the sealed chamber to create a vacuum within the sealed chamber.

Accordingly, the cancellous device is fully saturated with the solution, which may include but is not limited to a solution of DBM, in preparation for use as a bone graft. The solution promotes bone growth. Therefore, by saturating the cancellous device with the solution, bone growth adjacent the bone graft is improved. The vacuum container removes the air from the voids of the cancellous device, thereby allowing the solution to fill the voids and saturate the cancellous device.

The above features and advantages and other features and advantages of the present invention are readily apparent from the following detailed description of the best modes for carrying out the invention when taken in connection with the accompanying drawings.

### BRIEF DESCRIPTION OF THE DRAWINGS

FIG. 1 is a schematic cross sectional view of a vacuum container for saturating a cancellous device with a solution of Demineralized Bone Matrix (DBM) for use as a bone graft, showing an upper portion of the vacuum container detached from a lower portion of the vacuum container.

FIG. 2 is a schematic cross sectional view of the vacuum container showing the upper portion attached to the lower portion, and a vacuum creating device removing air from within a sealed chamber of the vacuum container.

### DETAILED DESCRIPTION

Those having ordinary skill in the art will recognize that terms such as “above,” “below,” “upward,” “downward,” “top,” “bottom,” etc., are used descriptively for the figures, and do not represent limitations on the scope of the invention, as defined by the appended claims.

Referring to the Figures, wherein like numerals indicate like parts throughout the several views, a vacuum container is generally shown at 20. The vacuum container 20 is configured for saturating a cancellous device 22 with a solution for use as a bone graft. The solution includes a bone growth promoting substance, such as but not limited to Demineralized Bone Matrix (DBM), morphogenic proteins, or stem cells. The solution may be in a liquid form, a gaseous form, or a powder form. While the written description below describes the solution as a solution of DBM, it should be appreciated that the solution may be comprised of one or more substances other than the DBM. Accordingly, the solution is not limited to a solution of DBM. As used herein, the term cancellous is defined as having a spongy or porous internal structure defining a plurality of voids. The cancellous device 22 may include a patient's own bone material, cadaveric bone material, a synthetic bone material, or some other material suitable for use as a bone graft. As used herein, the term Demineralized Bone Matrix (DBM) is defined as an allograft or autograft bone material having all inorganic bone minerals removed, leaving only the organic collagen bone matrix. The DBM includes growth properties that promote bone growth within the human body.

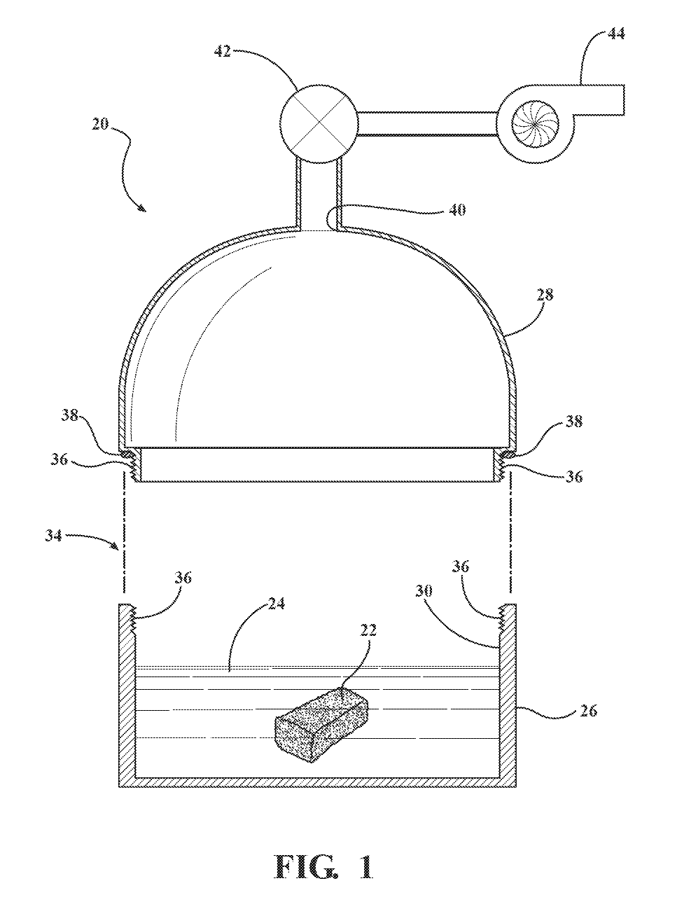

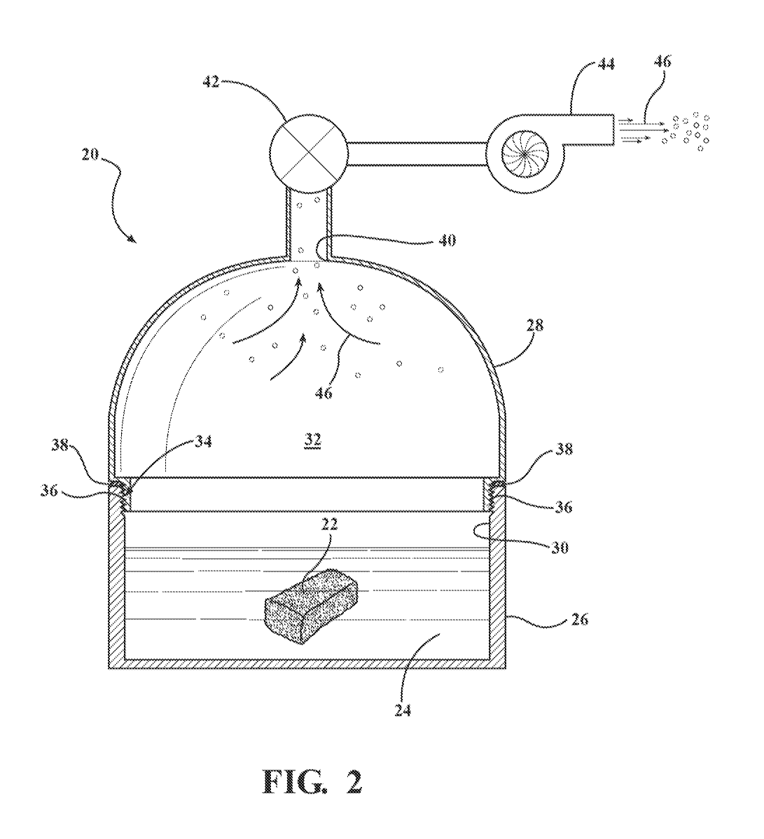

Referring to FIGS. 1 and 2, the vacuum container 20 includes a lower portion 26 and an upper portion 28. The lower portion 26 defines a reservoir 30 that is sized and shaped to contain the cancellous device 22 fully immersed within the solution of DBM 24. The upper portion 28 is removably attached to the lower portion 26. As shown in FIG. 1, the upper portion 28 is removable from the lower portion 26 to expose the reservoir 30 of the lower portion 26 to allow placement of the cancellous device 22 and the solution of DBM 24. When attached to the lower portion 26, as shown in FIG. 2, the upper portion 28 is disposed in sealing engagement with the lower portion 26 to define a sealed chamber 32 therebetween.

The vacuum container 20 includes an attachment mechanism 34 that interconnects the lower portion 26 and the upper portion 28. The attachment mechanism 34 may include any suitable device capable of securely attaching the upper portion 28 and the lower portion 26. For example, the attachment mechanism 34 may include threads 36 formed into both the upper portion 28 and the lower portion 26 and defining a threaded connection interconnecting the lower portion 26 and the upper portion 28 in threaded engagement. Alternatively, the attachment mechanism 34 may include one or more clamps (not shown) disposed about the periphery of the vacuum container 20 and configured for clamping the upper portion 28 and the lower portion 26 together. It should be appreciated that the attachment mechanism 34 may include some other device and/or configuration incorporated into the upper portion 28 and/or the lower portion 26 not described herein nor shown in the drawings that is capable of attaching the upper portion 28 and the lower portion 26.

The vacuum container 20 further includes a seal 38 disposed between the lower portion 26 and the upper portion 28 for sealing therebetween. The seal 38 may be attached to either the upper portion 28 or the lower portion 26, and seals between the upper portion 28 and the lower portion 26 to prevent the infiltration and exfiltration of fluids and/or gases into the sealed chamber 32 of the container when the upper portion 28 is attached to the lower portion 26. The seal 38 may include any suitable device, such as but not limited to an elastomeric o-ring, gasket, or other similar device.

The upper portion 28 of the vacuum container 20 defines an opening 40 extending therethrough into the sealed chamber 32. Preferably, and as shown, the opening 40 is defined by an upper wall of the upper portion 28. However, it should be appreciated that the opening 40 may be disposed on a side wall of the upper portion 28. A valve 42 is coupled to the upper portion 28 and disposed in fluid communication with the opening 40. The valve 42 is operable to open and close fluid communication through the opening 40. Accordingly, the valve 42 is moveable between an open position allowing fluid communication through the opening 40, and a closed position blocking fluid communication through the opening 40 and sealing the sealed chamber 32.

A vacuum creating device 44 is coupled to the valve 42 and disposed in fluid communication with the valve 42. The vacuum creating device 44 is operable to remove air from within the sealed chamber 32 to create a vacuum within the sealed chamber 32. As such, with the valve 42 disposed in the open position, the vacuum creating device 44 may be operated to remove the air from within the sealed chamber 32, generally represented by the flow arrow 46 shown in FIG. 2, after which, the valve 42 may be moved into the closed position to close the opening 40 and seal 38 the sealed chamber 32, thereby maintaining the vacuum in the sealed chamber 32. The vacuum creating device 44 may include, but is not limited to, a syringe or a vacuum pump. It should be appreciated that the vacuum creating device 44 may include some other device capable of removing the air from within the sealed chamber 32.

A method of preparing the bone graft is also provided. The method includes preparing the solution of bone growth promoting material, such as the solution of DBM 24. The solution of DBM 24 may be prepared in any manner suitable for sue with a bone graft, and is dependent on the specific substance and form of the bone growth promoting material used. For example, the solution may be prepared by suspending stem cells in amniotic fluid.

Once the solution of DBM 24 is prepared, the cancellous device 22 and the solution of DBM 24 are placed within the reservoir 30 of the lower portion 26, and the cancellous device 22 is fully submerged within and surrounded by the solution of DBM 24. When the cancellous device 22 is fully immersed within the solution of DBM 24, the upper portion 28 of the vacuum container 20 is attached to the lower portion 26 of the vacuum container 20 to define the sealed chamber 32 therebetween.

Once the upper portion 28 is attached to the lower portion 26, the valve 42 is opened, and the vacuum creating device 44 is engaged to remove the air from within the sealed chamber 32 through the opening 40 in the upper portion 28, thereby creating a vacuum therein. It should be appreciated that the vacuum creating device 44 must operate for a time period sufficient to draw the air out of the voids of the cancellous device 22, and through the solution of DBM 24. By removing the air from within the sealed chamber 32 and forming the vacuum therein, the air from the plurality of voids in the cancellous device 22 is also removed. Removing the air from the voids in the cancellous device 22, while the cancellous device 22 is immersed in the solution of DBM 24, allows the solution to fill the voids and fully saturate the cancellous device 22.

Once the air is removed from the sealed chamber 32, the valve 42 may be closed to seal 38 the opening 40 to maintain the vacuum in the sealed chamber 32. The vacuum in the sealed chamber 32 may be maintained for a time period sufficient to allow the solution of DBM 24 to fully saturate the cancellous device 22. It should be appreciated that the time period required to allow the solution of DBM 24 to infiltrate into the voids of the cancellous device 22 may depend upon the size and consistency of the cancellous device 22, and upon the properties of the solution of DBM 24. Once adequate time has passed to fully saturate the cancellous device 22, the valve 42 may be opened to release the vacuum within the sealed chamber 32, thereby allowing the upper portion 28 to be removed from the lower portion 26, and the saturated cancellous device 22 to be removed from the reservoir 30 and used as a bone graft.

While the method is described above using the vacuum container 20 shown in the Figures, it should be appreciated that the method may be practiced with any container capable of supporting the cancellous device 22 in a solution of bone growth promoting material, and forming a vacuum therein, such as but not limited to a specially configured syringe. Accordingly the method described and claimed herein is not limited to use with the vacuum container 20 described and claimed herein.

The detailed description and the drawings or figures are supportive and descriptive of the invention, but the scope of the invention is defined solely by the claims. While some of the best modes and other embodiments for carrying out the claimed invention have been described in detail, various alternative designs and embodiments exist for practicing the invention defined in the appended claims.

1. A method of preparing a bone graft, the method comprising:
immersing a cancellous device defining a plurality of voids within a solution of bone growth promoting material; and
removing air from the plurality of voids in the cancellous device while the cancellous device is immersed in the solution to allow the solution to fill the voids and saturate the cancellous device.

2. A method as set forth in claim 1 wherein the solution of bone growth promoting material includes one of a Demineralized Bone Matrix (DBM) solution, a morphogenic protein solution, or a stem cell solution.

3. A method as set forth in claim 1 wherein immersing the cancellous device within the solution includes placing the cancellous device and the solution into a container.

4. A method as set forth in claim 3 wherein immersing the cancellous device within the solution is further defined as immersing the cancellous device within the solution such that the cancellous device is completely submerged and surrounded by the solution.

5. A method as set forth in claim 3 further comprising closing the container to define a sealed chamber, with the cancellous device immersed in the solution within the sealed chamber.

6. A method as set forth in claim 5 wherein removing the air from the plurality of voids in the cancellous device includes removing the air from the sealed chamber to create a vacuum in the sealed chamber of the container.

7. A method as set forth in claim 1 further comprising preparing the solution of DBM.

8. A method as set forth in claim 1 wherein the cancellous device includes cadaveric bone.

9. A method of saturating a cancellous device with a solution of Demineralized Bone Matrix (DBM) for use as a bone graft, the method comprising:
placing the cancellous device and the solution of DBM within a reservoir of a container, with the cancellous device fully immersed within and surrounded by the solution of DBM;
sealing the container to define a sealed chamber, with the cancellous device and the solution of DBM disposed within the sealed chamber; and
removing air from the sealed chamber to form a vacuum in the sealed chamber, wherein air trapped within a plurality of voids of the cancellous device is removed so that the solution of DBM may fill the plurality of voids and saturate the cancellous device.

10. A method as set forth in claim 9 further comprising preparing the solution of DBM.

11. A method as set forth in claim 9 wherein removing air from the sealed chamber includes removing air from the sealed chamber through an opening in the container with a vacuum creating device.

12. A method as set forth in claim 11 further comprising closing a valve to seal the opening after removing the air from the sealed chamber to maintain the vacuum in the sealed chamber.

13. A vacuum container for saturating a cancellous device with a solution of Demineralized Bone Matrix (DBM) for use as a bone graft, the vacuum container comprising:
a lower portion defining a reservoir configured for containing the cancellous device fully immersed within the solution of DBM;
an upper portion removably attached to the lower portion and disposed in sealing engagement with the lower portion to define a sealed chamber, wherein the upper portion defines an opening into the sealed chamber;
a valve coupled to the upper portion and operable to open and close fluid communication through the opening; and
a vacuum creating device in fluid communication with the valve and operable to remove air from within the sealed chamber to create a vacuum within the sealed chamber.

14. A vacuum container as set forth in claim 13 wherein the vacuum creating device includes one of a syringe or a vacuum pump.

15. A vacuum container as set forth in claim 13 further comprising a seal disposed between the lower portion and the upper portion for sealing therebetween.

16. A vacuum container as set forth in claim 13 further comprising an attachment mechanism interconnecting the lower portion and the upper portion.

17. A vacuum container as set forth in claim 16 wherein the attachment mechanism includes a threaded connection interconnecting the lower portion and the upper portion in threaded engagement.
